(19) United States
(12) Patent Application Publication
(10) Pub. No.: US 2026/0125733 A1
(43) Pub. Date: May 7, 2026

(54) SEALING MICROLID FOR TESTING VOLATILE AGENTS

(71) Applicant: CESKA ZEMEDELSKA UNIVERZITA V PRAZE, Praha-Suchdol (CZ)

(72) Inventors: Ladislav KOKOSKA, Praha-Suchdol (CZ); Marketa HOUDKOVA, Sruby (CZ)

(21) Appl. No.: 19/117,849

(22) PCT Filed: Oct. 6, 2022
(86) PCT No.: PCT/IB2022/059531

§ 371 Date: Apr. 2, 2025

(51) Int. Cl. C12Q 1/18

(52) U.S. Cl. CPC C12Q 1/18

(57) ABSTRACT

A sealing microlid (novel microplate sealing lid) designed to evaluate the biological effects of volatile agents in vapour phase in combination with a microplate is disclosed. The sealing microlid includes a plate having a surface, a plurality of flanges extending from the surface, and a protruding opening tab. The flanges of the sealing microlid serve as reservoirs for growth medium containing organisms or cells, which will grow on it, and, at the same time, generate an airtight condition with the well of the microplate necessary for leakage-prevention of the volatile agents tested. The apparatus of the present invention allows quantitative, rapid, simple, labour-and cost-effective susceptibility testing of organism or cell cultures to volatile agents in vapour phase.

A microplate (microtiter plate), a flat plate with multiple wells used as small test tubes, is common labware used for numerous assays in genetics, chemistry, microbiology, and pharmacy. The most common well densities are 6, 12, 24, 48, 96, 384 or 1536 wells per plate. Each well of the microplate typically holds somewhere between tens of nanolitres to several millilitres of liquid. The wells are available in different shapes of bottom such as flat, U- and V-shaped and bottom with minimal rounded edges. Microplates are, in general, designed to be disposable and are manufactured from a variety of materials such as cyclic olefin copolymer, polypropylene, and polystyrene. They are typically produced in opaque white, opaque black, or translucent colours. There are also microplates constructed from solid pieces of glass and quartz for special applications. The standardization of the microwell plates is conducted by the Society for Biomolecular Sciences with the American National Standards Institute and Society for Laboratory Automation and Screening. Plate lids and seals are options for the protection of the well's contents from leakage, contamination, and evaporation during assay processing, incubation, or storage. They are available for a variety of plates, and in a variety of materials, many with specific applications, including storage, biological assays, microscopy, and cell culture (Markossian et al. 2004, Assay Guidance Manual, Eli Lilly & Company and NCATS, Bethesda). The minimum biofilm eradication concentration assay using a microplate/peg lid system for testing the susceptibility of biofilms to antimicrobial agents is an example of a patented (patents U.S. Pat. Nos. 6,051,423, 6,326,190, 6,410,256, 6,596,505 and 6,599,714) and commercially available product (Innovotech, Edmonton, Canada) based on a modified microplate lid.

Volatile agents, being compounds of low molecular weight and high vapour pressure at ambient temperature, are present in various matrices, including plant materials, from which they can be obtained, for example, in the form of essential oils. They are of great potential for the development of novel medicinal, pharmaceutical, food and agricultural products and technologies, such as inhalation therapy, active food packaging, and controlled-atmosphere storage. However, the industrial applications based on the most typical physico-chemical feature of these agents, which is volatility, have not been fully developed yet. One of the main reasons for this situation is the lack of efficient quantitative methods suitable for the high-throughput screening of the biological activity of the volatile agents. A disc volatilization method based on the evaporation of volatile agents from a solid matrix (e.g. paper disc) is the most commonly used assay; however, it is time- and labour-consuming and allows qualitative evaluation only. With the exception of several experimental apparatuses that are not commercially available, there is no special labware designed for testing the biological activity of volatile agents in vapour phase. Therefore, there remains a need for a novel device and method that will overcome these disadvantages (Houdkova & Kokoska, 2020, Planta Med. 86,822).

Recently, our team designed a high-throughput screening assay performed with microplates covered by lids with flanges designed to reduce evaporation. This broth microdilution volatilization method is suitable for the simple and rapid simultaneous determination of the antibacterial potential of volatile agents in liquid and vapour phases at different concentrations. It allows for the cost-and labour-effective high-throughput screening of volatile agents using commercially available microplates. However, since the assay described above is performed using serially produced microplates that are not designed for this purpose, the method suffers from several weaknesses. For example, clamps are necessary for fastening the plate and lid together and only a limited volume of agar can be applied on the lid, which could affect bacterial growth (Houdkova et al. 2017, Fitoterapia 118,56).

With the aim of overcoming the problems related to the assessment of the biological activity of volatile agents inherent in the prior art solutions, the present invention provides an apparatus for testing the effects of volatile agents on the growth of organisms or cells in vapour phase. The present invention utilizes a sealing microlid (novel microplate sealing lid) with flanges designed to carry growth medium for the cultivation of organisms or cells, which is placed onto the top of the standard microplate. The flanges of the sealing microlid serve two important functions. The first function is as a reservoir for the growth medium (e.g., agar) containing cells or (micro)organisms such as bacteria, yeast, and fungi, which will grow on it. The second function of the flange is to generate an airtight condition with well of the microplate, necessary for leakage-prevention of the volatile agents tested. The microplate receives the sealing microlid with flanges filled with growth medium (e.g. agar) and organism or cell culture in an airtight communication and retains a volatilization matrix (e.g. broth) with the tested volatile agent. Initially, the flanges of the sealing microlid receive the growth medium and cultures. Then, the volatilization matrix containing a volatile agent is added into the wells of the microplate. Alternatively, it may be desirous to use flanges as carriers of the volatilization matrices (e.g. through the insertion of paper discs containing volatile agents into the flanges). In this case, organism or cell cultures are grown in the wells of the plate. The sealing microlid allows for various agents to be simultaneously tested at different concentrations. Simultaneous susceptibility testing of different organisms or cells in one sealing microlid is also possible. The sealing microlid is allowed to incubate for a period of time in which the susceptibility of cultures to biocidal agents is tested. After this time period, the sealing microlid is removed from the microplate and the effectiveness of the biocides may be tested. Biological activity of volatile agents is then assessed in the sealing microlid and microplate and expressed as an inhibitory concentration (half maximal inhibitory concentration, minimum inhibitory concentrations, etc.). In combination with a conventional microplate, the apparatus of the present invention allows quantitative, rapid, simple, labour-and cost-effective susceptibility testing of organism and cell cultures to volatile agents simultaneously in liquid and vapour phase. Furthermore, the sealing microlid allows for high-throughput testing of volatile samples and provides accurate and reproducible results.

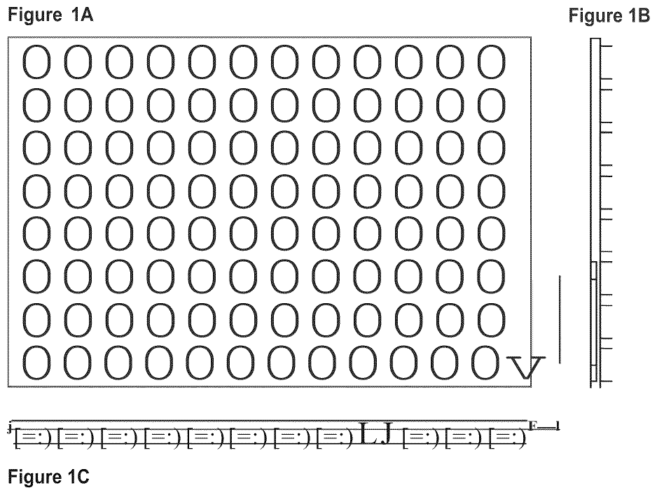

Referring now to FIG. 1, which shows an orthographic projection of the present invention. The sealing microlid includes a plate having a surface, a plurality of flanges extending from the surface, and a protruding opening tab. The preferably unitarily formed flanges extend from the surface of the sealing microlid and have a general hollow cylindrical geometry. Although shown as having a general hollow cylindrical shape, the projections may be formed having any appropriate geometry, for example, a hollow conical shape or any similar geometries depending on the shape of the microplate wells. The flanges may be formed, for example, in eight rows of twelve flanges in each row as shown in FIG. 1. In this configuration, the sealing microlid may be combined with a commonly available 96-well plate in order to form an airtight system for the susceptibility testing of organisms and cells to volatile agents in vapour phase. Although the sealing microlid is illustrated as containing 96 wells, it is contemplated that other numbers of flanges formed in a number of different geometrical patterns may be utilized. It shall be understood that the sealing microlid will be chosen such that the number and shape of the flanges which will correspond to the number and shape of wells and the geometrical pattern of the plate. Therefore, the present invention may be designed for use in combination with any plates used for the cultivation of organisms or cells, for example, with 24 and 48 well plates. The sealing microlid may be constructed from any bio-compatible material such as stainless steel, glass, and plastic. Preferably, the sealing microlid is formed from sold or flexible plastic, such as ethylene vinyl acetate, low-density poly-ethylene, polycarbonate, polyethylene, polypropylene, polystyrene, polyvinylchloride, silicon, and other similar materials. The sealing microlid may be formed having opaque or transparent characteristics, thereby allowing a user to view the organism or cell culture formation on the growth medium. Furthermore, the sealing microlid can be printed or embossed with a unique identifier for each flange, for example each column of a 96-flange sealing microlid can be identified by a number from 1-12 and each row of a 96-flange sealing microlid can be identified by a letter from A-H. Other labelling systems can also be used.

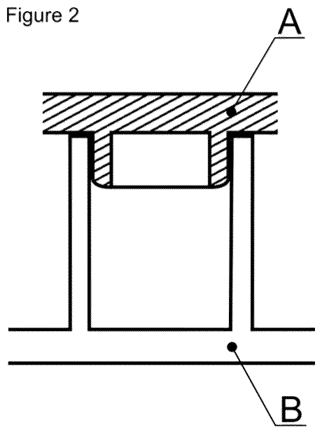

Referring now to FIG. 2, which shows a detailed side cross-sectional view of a single flange of the microlid sealing one well of a microplate. As described above and illustrated in the drawing, the sealing microlid is designed such that the plate will accept the sealing microlid thereby forming an airtight seal between the sealing microlid and the plate. Therefore, the size and shape of the sealing microlid flanges and microplate wells should ensure airtight conditions of the system. This airtight enclosure prevents the leakage of the volatile agents tested. As described earlier, the flanges form growth medium adherent sites on which cultures of organisms or cells may grow.

The preferred embodiments of the invention will now be described more particularly, with reference to the appended drawings, by way of illustration. Understanding that these drawings depict only the typical embodiments of the invention and are therefore not to be considered as limiting in its scope, the invention will be described and explained with additional specificity and detail through the use of the accompanying drawings in which:

FIG. 1 is an orthographic projection of the sealing microlid (novel microplate sealing lid) for a 96 well microplate designed to evaluate the biological effects of volatile agents in vapour phase, whereas part A is a top view and parts B and C are side views of an alternative embodiment of the present invention;

FIG. 2 is a side cross-sectional view of a single flange of the sealing microlid (A) sealing the well of the conventional microplate (B).

1-4. (canceled)

5. A method of susceptibility testing of organisms or cell cultures, comprising:
providing a microplate having a plurality of wells, the plurality of wells arranged in plural rows and plural columns;
providing a sealing microlid for the microplate, the sealing microlid comprising a plate having a substantially planar base structure and a plurality of flanges extending from a lower surface of the plate, the plurality of flanges arranged in plural rows and plural columns so that each of the plurality of flanges corresponds to one of the plurality of wells, each of the plurality of flanges adapted to air-tight seal and tightly fit into the corresponding well on the microplate;
filling each of the plurality of wells with a growth medium containing organisms or cell cultures;
adding a volatile agent to each of the plurality of flanges;
positioning the sealing microlid over the microplate so that each of the plurality of flanges seals the corresponding well on the microplate; and
after an incubation period, assessing an effect of the volatile agent on growth of the organisms or cell cultures in each of the plurality of wells.
